(19) United States
(12) Patent Application Publication
(10) Pub. No.: US 2008/0319343 A1
(43) Pub. Date: Dec. 25, 2008

(54) Fetal blood sampling device

(76) Inventor: Michael Milano, Livingston, NJ (US)

Correspondence Address: KLAUBER & JACKSON, 411 HACKENSACK AVENUE, HACKENSACK, NJ, 07601, US

(21) Appl. No.: 12/006,829

(22) Filed: Jan. 4, 2008

Related U.S. Application Data
(60) Provisional application No. 60/936,868, filed on Jun. 22, 2007.

(51) Int. Cl. A61B 5/15

(52) U.S. Cl. 600/573

(57) ABSTRACT

A fetal scalp attachable blood-sampling device, which includes an annular tube the outer wall and the axial opening of which generally taper from an enlarged proximal end to an opposed smaller distal end. An annular ring-like space is defined in the wall of the tube and surrounds the distal end of the tube and is also open at its distal end. The tube has a continuous outer wall which smoothly engages with the outside of the annular ring-like space without presenting any sharp corners. The device is insertable by the attending physician into the vaginal canal of a patient for engaging at the distal end of the device with the scalp of a fetus while the patient is in labor. An air suction tube extends within the tube wall from a point external to the device and connects to the interior of the annular sleeve. Accordingly, a vacuum applied to the suction tube causes the distal open end of the sleeve to securely engage the fetus scalp to enable blood sampling by the physician via the internal tube. A handle is secured to the tube at the physician accessible end of the device to facilitate emplacement of the device by the physician into the vaginal canal of the patient and its proper engagement with the fetal scalp. The handle is mounted at the outer surface of the tube as to be angled with respect to the axis of said device in the direction toward the rear of the device, to further facilitate use of the handle during manipulation of the device by the physician. Vacuum application means are connected to the air suction tube at a point within the convenient reach of the physician while the physician is emplacing the device in contact with the fetus scalp. This enables the physician to control application of suction at the distal end of the device as it is emplaced at the fetus scalp.

### REFERENCE TO RELATED APPLICATIONS

The present patent application is based upon and hereby claims priority to provisional patent application Ser. 60/936,868, filed Jun. 22, 2007.

### FIELD OF INVENTION

This invention relates generally to medical and surgical apparatus, and more specifically relates to a blood sampling device which is attachable to the scalp of a fetus within a patient in labor, in order that blood samples from the fetus may be obtained to evaluate whether the fetus is in a stressed condition.

### BACKGROUND OF INVENTION

During the course of an infant delivery there is often a degree of distress occurring, which requires monitoring of the fetal condition. A fetal heart monitor is well known to be used for this purpose. Basically if it appears from the monitoring that the fetus is in danger, then a Cesarean section is used for delivery.

In order to further monitor the fetus a kit is commercially available which includes a conical device, which is inserted into the patient's vagina and through the cervical opening in order to contact (beyond the cervical opening) the fetal scalp. A small pinprick is then made in the fetal scalp (via a knife or lance included with the kit) and a capillary tube is used to extract a drop or two of fetal blood so that it can be measured for its pH and thereby the oxygen content determined. If the pH confirms fetal acidosis, then fetal oxygenation is compromised and a C-section would be performed. In practice, however, use of the device just described is difficult and cumbersome, especially because of the difficulty arising in firmly contacting the fetal scalp with the open end of the cone—and then maintaining the contact.

In U.S. Pat. No. 3,685,509 an endoscope is disclosed for fetal blood sampling which considerably improves upon the mentioned kit device. In the device of the '509 patent a medical instrument is disclosed with two tubes arranged to define an annular space there between for application of suction to adhere the device to a body part while an associated instrument is housed within or passed through the inner tube. This finds particular application to endoscopes, especially amniscopes for fetal blood sampling, with one of the tubes being shorter than and preferably outside the longer main tube at its distal end, with a third tube of smaller order of cross-section passing back along the main tube towards its proximal end and having means at its proximal end for connecting the annular space with a vacuum source. A lance of similar length to but significantly smaller diameter than said first tube is disposed within and longitudinally along the interior of the first tube, and is translatable axially with respect to said first tube so that the end of the lance may be moved beyond the first tube to puncture a fetus held across the distal end of said first tube by suction applied to said annular space. A sample-extracting fourth tube of smaller diameter than the other tubes is disposed to extend longitudinally along said first tube to terminate at the distal end of the interior thereof to draw off a fetal blood sample from the puncture.

Careful consideration of the device of the U.S. Pat. No. 3,685,509 patent reveals a series of difficulties presented by the design:

1. In the '509 invention, the exterior transition between the main tube and the second annular tube is defined by a 90° angle. This results in safety issues. The walls of the pregnant vagina are comprised of numerous folds or rugae, which are highly vascular. It should be appreciated that the mentioned transition area, of necessity, would be inserted deep into the vaginal canal and would have to traverse the entire length of the vagina to be removed. Upon withdrawal of the instrument, the edge of this right-angled transition could easily get entangled in one of these folds, resulting in a tearing of the tissue, which is quite brittle in pregnancy. This would result in a dangerous hemorrhage.

2. The prior art '509 patent describes a tube exterior to the main cone, with an angled bulbous end portion to connect to a flexible suction line. This added irregularity of tubing and bulbous end portion externally presents difficulties and can generate trauma during both the introduction and removal of the instrument. Additionally, in a particularly deep vagina, the angled end portion may have to enter into the vagina by necessity and disappear from view inside the vaginal canal. This would render the connection between the angled bulbous end and suction tubing inaccessible. In the event that the suction tubing became loose at the connection to the bulbous end, the operator would be unable to tighten that connection without removing the entire instrument and beginning the procedure over again. Furthermore, in this scenario, the protuberant piece has the potential to become ensnared in the vaginal mucosa upon withdrawal and thus could also result in tearing and bleeding.

3. The prior art '509 patent makes no specific accommodation to provide a suction source under the precise, direct control of the attending physician. Instead, it describes in a general manner a “fitting for connection to a flexible suction line and associated evacuation pump.” This would have to be accomplished by connection of a wall suction unit to the device by a second party. However, it is critical for ease of use, given the emergent conditions under which the procedure is done, that an integral pump, controlled by the physician, be part and parcel of the device. It must be appreciated that when inserting the cone to establish firm contact with the fetal scalp, the cervix will often slip down between the cone and the fetal head, thereby obscuring visualization of the operative field. To avoid trapping the cervix in the suction ring requires instantaneous application of suction at the proper time while the head is in view of the physician and this application of suction force must be under the direct control of the physician and not by a second party. It is imperative to avoid any time delays caused by the very nature of verbally communicating an order to the second party. Application of suction too soon or too late would likely attach the cone to the cervix.

### SUMMARY OF INVENTION

Now in accordance with the present invention a fetal scalp attachable blood sampling device is provided which substantially improves upon the type of apparatus disclosed in the prior art U.S. Pat. No. 3,685,509 patent, and overcomes the deficiencies of that earlier invention that have been considered in the forgoing discussion.

Pursuant to the instant invention a fetal scalp attachable blood sampling device is provided which includes an annular tube, the outer wall and the axial opening of which generally taper from an enlarged proximal end accessible to the attending physician, to an opposed smaller distal end. An annular ring or ring-like space is formed in the wall of the tube at its distal end and like the tube is also open at its distal end. The annular tube has a continuous outer wall which smoothly engages with the outside of the annular ring without presenting any sharp corners. The device is insertable by the attending physician into the vaginal canal of a patient for engaging at the distal end of the device with the scalp of a fetus while the patient is in labor. An air suction tube extends in an axial direction within the wall of the annular tube from a point external to the device and connects to the interior space of the annular ring. Accordingly, a vacuum applied to the suction tube causes the distal open end of the sleeve to securely engage the fetus scalp to enable blood sampling by the physician via the internal opening of the tube.

A handle is secured to the outside of the annular tube at the physician accessible proximal end of the device to facilitate emplacement of the device by the physician into the vaginal canal of the patient and its proper engagement with the fetal scalp. Preferably the handle is mounted at the tube as to be angled with respect to the axis of said device in the direction toward the rear of the device, to further facilitate use of the handle during manipulation of the device by the physician.

Vacuum application means manually actuated by the physician are connected to the air suction tube at a point within the convenient reach of the physician while the physician is emplacing the device in contact with the fetus scalp. This enables the physician to accurately control application of suction at the distal end of the device as it is emplaced at the fetus scalp.

The invention may further include a light holding compartment formed at the wall of the enlarged open end of the annular tube for enabling insertion of a light source to illuminate the patient and fetal regions where the physician is inserting and positioning the device. The compartment is open at its far end to facilitate insertion of the light source. Use of the light source enables accurate illumination at the portion of the fetal scalp which is to be pierced by the lance, and facilitates subsequent rapid and accurate positioning of a capillary tube to draw off the blood sample from the pierced point of the scalp.

### BRIEF DESCRIPTION OF DRAWINGS

The invention is diagrammatically illustrated, by way of example, in the drawings appended hereto, in which:

FIG. 1 is a schematic transverse sectional view of a fetal scalp attachable blood sampling device in accordance with the invention;

FIG. 1A is a schematic rear (proximal) end view of the device of FIG. 1; and

FIG. 2 is a partial side elevational view of a manually controlled vacuum device that may be used to control application of a vacuum to the suction tube portion of the device in FIG. 1.

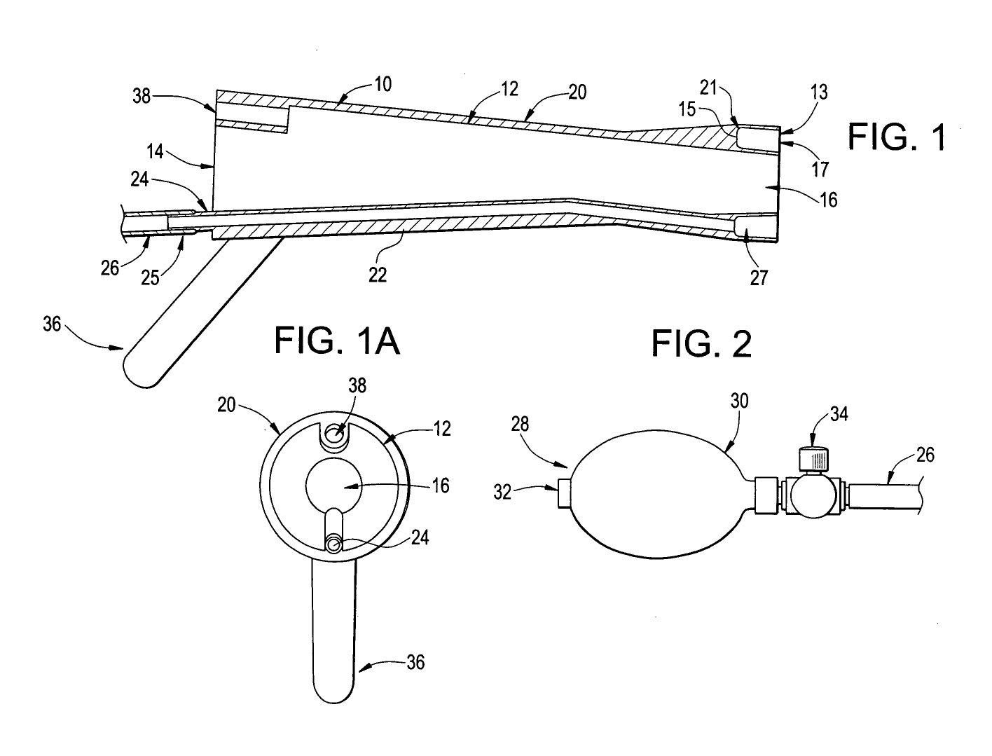

### DESCRIPTION OF PREFERRED EMBODIMENT

The blood sampling device 10 is seen to include a generally cone-shaped annular tube 12 which is open at both its enlarged proximal end 14 and its smaller distal end 16. Tube 12 is generally convergent from its end 14 to end 16. At its distal end 16 an annular ring or ring-like space 13 is formed or embedded in wall 22 of tube 12. Ring 13 is closed at its rear 15, but open at its distal end 17. The outside wall 20 of tube 13 while also generally converging toward the distal end of device 10, is seen to be slightly and smoothly diverged at 21 toward its distal end so that it may be smoothly formed about the ring 13 without any right angle or sharp edges or discontinuities being presented at the lateral sides of device 10.

At one side of device 10 it is further seen that a vacuum draw tube 24 extends from an external point 25 rearward of the tube 12, and passing in an axial direction through the length of wall 22 is connected to the rear of ring 13 so as to be in communication with the interior volume 27 of ring 13. A piece of flexible tubing 26 is connected to the rear of tube 24 and is in turn connected rearwardly to the vacuum drawing and control device 28 shown in FIG. 2. Device 28 comprises a hand bulb 30 with a one-way exhaust valve 32.

A control valve 34 when manually opened allows air to return to the tube 24 to release the suction provided at the distal end of device 10 via the open end 17 of annular ring 13. Upon the device 10 being properly positioned against the scalp of the fetus, the physician draws a slight vacuum via hand bulb 30, which creates a firm seal with the scalp of the fetus. A porous sponge material may be provided at the distal end of the annular ring 13 through which air may be effectively drawn while at the same time providing a relatively soft surface for engaging with the fetus scalp.

A handle 36 is affixed to the rear of tube 12 to facilitate manipulation of the device 10. This handle is affixed as to be angled toward the rear of device 10, typically at an angle of about 45 degrees. A light source-receiving compartment 38 is also provided at the rearward wall of tube 12. Compartment 38 is open at both its ends. A light source can be inserted in the compartment 38 in order to illuminate the scalp area to be acted upon. A surgical lance can be passed through the central interior of tube 12 to engage and lightly puncture the skin at the illuminated scalp of the fetus to produce the desired blood sample, which is then collected for analysis with a capillary tube, which can also be passed through the central interior of tube 12. Using the incident illumination from compartment 38 to help guide and position the lance and/or capillary tube can add to the accuracy and speed of performing the sampling procedure.

Recapitulating the advantages of the present invention: The addition of handle 36 to the proximal or outer end of the device 10 addresses the fact that the device is cumbersome to manipulate into position against the fetal head. This relates to the cervical opening usually lying at an oblique angle to the long axis of the vagina and is further complicated by the redundant folds of the gravid vagina. Therefore, the use in the present invention of a simple handle at the bottom of the proximal end of the cone, coming off at a 45 degree angle down (and thus out of the line of sight). The handle 36 is about as long as the length of a first, approximately 8 centimeters, and enables the physician to exert the proper leverage to angle the distal end of the device into place. It functions similar to the handle on a vaginal speculum, but is of a simpler design. This improves the functionality of the instrument.

The prior art of the '509 patent describes a tube which is exterior to the main cone, with an angled bulbous end portion to connect to a flexible suction line. The present invention places this tubing inside the wall 22 of tube 12, which maintains a smoother external surface of the conical form of tube 12, without this added irregularity of tubing and bulbous end portion externally. This facilitates both the introduction and removal of the instrument, as the smoother surface will glide in and out in a more efficient (and less traumatic) manner. Additionally, in a particularly deep vagina, the angled end portion of the prior art '509 patent may have to enter into the vagina by necessity and disappear from view inside the vaginal canal. This would render the connection between the angled bulbous end and suction tubing inaccessible. In the event that the suction tubing became loose at the connection to the bulbous end, the physician would be unable to tighten that connection without removing the entire instrument and beginning the procedure over again. Furthermore, in this scenario, the protuberant piece has the potential to become ensnared in the vaginal mucosa upon withdrawal and thus could also result in tearing and bleeding. The connection in the present invention, at tubing 25 to generate suction at the distal end is housed internally to the tube 12, and the attachment to the suction device 28 is at the back or proximal end of the tube 12 and in full control of the physician. The vacuum drawing device 28 in the present invention is thus under the precise, direct control of the attending physician.

In the prior art of the '509 patent, the exterior transition between the main tube and the second annular tube is defined by a 90° angle. The present invention improves upon this by creating a tapered transition of the exterior of the device between the two tubes. This is an important safety consideration. The walls of the pregnant vagina are comprised of numerous folds or rugae, which are highly vascular. The transition area, of necessity, would be inserted deep into the vaginal canal and to be removed would have to traverse the entire length of the vagina. Upon withdrawal of the prior art instrument, the edge of the right-angled transition could easily get entangled in one of these folds, resulting in a tearing of the tissue, which is quite brittle in pregnancy. This would result in a dangerous hemorrhage. By tapering the transition between the two tubes, no such entanglement can occur.

While the present invention has been set forth in terms of specific embodiments thereof, it will be appreciated that in view of the present disclosure numerous variations upon the invention are now enabled to those skilled on the art, which variations yet reside within the present teachings. Accordingly the invention is to be broadly construed, and limited only by the scope and spirit of the disclosure and of the claims now appended hereto.

1. A fetal scalp attachable blood sampling device for use by an attending physician at a patient in labor, comprising:
(a) an annular tube, the outer wall and the axial opening of which generally taper from an enlarged proximal end to an opposed smaller distal end;
(b) an annular ring-like space being defined in the wall of the said tube and surrounding the distal end of said tube, and being open at its distal end;
(c) the wall of said tube having a continuous outer surface which smoothly engages with the outside of said annular ring-like space without presenting any sharp corners;
(d) the said tube being insertable by the attending physician into the vaginal canal of said patient for passing into the cervical opening and engaging at the distal end of said device with the scalp of a fetus while the patient is in labor;
(e) an air suction tube extending within the wall of said tube from a point external to said device to said annular sleeve; said suction tube being in gas communication with the interior volume of said annular ring-like space, whereby a vacuum applied to said suction tube causes the distal open end of said ring-like space to securely engage the fetus scalp to enable blood sampling by said physician via the interior of the internal tube; and
(f) handle means secured to the outer surface of said tube toward the physician accessible end of said device to facilitate emplacement of the device by the physician into the vaginal canal of the patient and its proper engagement with the fetal scalp.

2. A device in accordance with claim 1, further including vacuum application means connected to said air suction means at a point within the convenient reach of the physician while emplacing the device in contact with the fetus scalp, for enabling the physician to control application of suction at the distal end of said device as it is emplaced at the fetus scalp.

3. A device in accordance with claim 2, further including a light holding compartment formed in the wall of said tube at the enlarged open end of the tube for enabling insertion of a light source to illuminate the patient and fetal regions where the physician is inserting and positioning the said device.

4. A device in accordance with claim 3, wherein the said compartment is open at its end remote from the enlarged end of said tube, to facilitate insertion of a said light source in order to achieve accurate illumination at the portion of the fetal scalp which is to be pricked by the lance, and to facilitate subsequent rapid and accurate positioning of a capillary tube to draw off the blood sample from the pricked point of the scalp.

5. A device in accordance with claim 1, wherein said handle is mounted at said tube as to be angled with respect to the axis of said device in the direction toward the rear of the device, to further facilitate use of the handle during manipulation of the device by the physician.
